(19) United States
(12) Patent Application Publication
(10) Pub. No.: US 2024/0424031 A1
(43) Pub. Date: Dec. 26, 2024

(54) COMPOSITION COMPRISING PASTEURIZED AKKERMANSIA FOR THE TREATMENT OR PREVENTION OF IBS-RELATED ANXIETY

(71) Applicant: THE AKKERMANSIA COMPANY, Mont-Saint-Guibert (BE)

(72) Inventors: Patrice CANI, Mont-Saint-Guibert (BE); Amandine BROCHOT, Mont-Saint-Guibert (BE)

(21) Appl. No.: 18/684,682

(22) PCT Filed: Aug. 2, 2022
(86) PCT No.: PCT/EP2022/071663

§ 371 Date: Feb. 18, 2024

(30) Foreign Application Priority Data
Aug. 19, 2021 (BE) BE2021/5660

(51) Int. Cl. A61K 35/74; A61K 45/06; A61P 25/22

(52) U.S. Cl. CPC A61K 35/74; A61K 45/06; A61P 25/22

(57) ABSTRACT

Disclosed are compositions comprising pasteurized Akkermansia for the treatment or prevention of IBS-related anxiety. Further disclosed is the use of the composition for promoting mental health or reducing anxiety, non-depressive mood disorders or stress in IBS patients. A further object, is the use of the composition for improving the gut-brain axis.

### FIELD OF THE INVENTION

The present invention relates to a composition comprising pasteurized Akkermansia muciniphila for the treatment or prevention of IBS-related anxiety.

### BACKGROUND

WO2018012834A1 to Korea Research Institute discloses Akkermansia muciniphila for preventing or treating degenerative brain diseases and for improving exercise control ability, cognitive ability and memory in Parkinson's disease or Alzheimer's disease. However, no pasteurized Akkermansia is disclosed. Moreover, the indication neurodegenerative disease is not disclosed in conjunction with IBS.

WO2017042347A1 to UCL and University of Wageningen discloses the use of pasteurized Akkermansia for IBS. However, the use for treating IBS-related anxiety is not disclosed. This international patent application was filed on Sep. 9, 2016 and has been granted in Europe.

WO2017178496A1 to University of Wageningen discloses Akkermansia glycaniphilus for use in preventing or treating irritable bowel syndrome (IBS), neurodegenerative diseases, or depression. However, no pasteurized A. glycaniphila is disclosed. Moreover, the indication depression is not disclosed in conjunction with IBS.

WO 2021/008149 to Jiangsu province hospital of Chinese medicine discloses the application of pasteurized Akkermansia Muciniphila bacteria in the preparation of drugs or health products for preventing and treating depression. However, this document does not disclose the use of Akkermansia muciniphila for the treatment of anxiety disorders which are clearly different from depressive disorders. Both mental disorders are on the rise since the COVID-19 pandemic.

For example, Santomauro et al. clearly distinguish anxiety disorders from depressive disorders, see case definitions on page 1702, left column, first paragraph, Santomauro, Global prevalence and burden of depressive and anxiety disorders in 204 countries and territories in 2020 due to the COVID-19 pandemic, www.thelancet.com Vol 398, Nov. 6, 2021. Santamauro used case definitions for major depressive disorder and anxiety disorders used within the Global Burden of Diseases, Injuries, and Risk Factors Study (GBD) framework. These definitions adhere to criteria presented in the Diagnostic and Statistical Manual of Mental Disorder fourth edition text revision (DSM-IV-TR) and the tenth International Classification of Diseases and Related Health Problems (ICD-10).

Therefore, there is still an urgent need to provide improved compositions for the treatment or prevention of IBS-related anxiety.

### SHORT DESCRIPTION OF THE INVENTION

The present inventors have surprisingly found that a composition comprising pasteurized Akkermansia muciniphila reduces IBS-related anxiety already after a very short administration time of around four days.

Accordingly, a first aspect is a composition, comprising pasteurized Akkermansia, in particular Akkermansia muciniphila for use in the prevention or treatment of anxiety, stress or non-depressive mood disorders, wherein the anxiety, stress or non-depressive mood disorders are related to Irritable Bowel Syndrome (IBS). Such disorder may also be a comorbidity of IBS.

In one embodiment, the mood disorder is not depressive mood disorder and in particular not major depressive mood disorder.

In one embodiment, the composition comprising pasteurized Akkermansia, in particular Akkermansia muciniphila is used in the prevention or treatment of anxiety or stress, wherein the anxiety or stress are related to IBS.

In one embodiment, the composition comprising pasteurized Akkermansia is used in the prevention or treatment of non-depressive mood disorders, wherein the non-depressive mood disorders are related to IBS.

In another aspect, the pasteurized Akkermansia is Akkermansia muciniphila.

In another aspect, the Akkermansia, in particular the Akkermansia muciniphila is not pasteurized.

In another aspect, the composition of the invention is used for the treatment or prevention of the early stage of anxiety, stress or non-depressive mood disorder.

In another aspect, the early stage is the onset of anxiety, stress or non-depressive mood disorder, for example 1, 2, 3, or five days after its beginning.

In another aspect, the composition is administered for one week or longer, for two weeks or longer or even permanently.

In another aspect, the composition is administered sufficiently in advance of a stress-provoking event to prevent, control or reduce the development of anxiety, stress or non-depressive mood disorder to a subject.

In another aspect, pasteurized Akkermansia is administered for 2 to 10 days, preferably for 3 to 7 days and even more preferably for 4 to 6 days.

In another aspect, the pasteurized Akkermansia is administered in an amount from 1.10⁴ to 1.10¹² cells per day, more preferably from 1.10⁵ cells to 1.10¹¹ cells per day, and even more preferably from 1.106 to 5.10¹⁰ cells per day.

In another aspect, the pasteurized Akkermansia is administered in an amount from 1.10⁸ to 5.10¹⁰ cells per day.

In another aspect, the composition further comprises one or more ingredients chosen from group consisting of probiotic, bacteria, yeast, microorganisms, prebiotic or a combination thereof.

In another aspect, the composition further comprising a mineral or a vitamin or a combination thereof.

In another aspect, the composition further comprises a pharmaceutically acceptable carrier or a food grade carrier.

In another aspect, the composition is orally administered.

In another aspect, the composition is a cosmetic composition, a nutritional composition, a food product, a dietary complement, a medical food or a medicament.

In another aspect, the composition further comprises a plant extract.

In another aspect, the plant extract is chosen from the group consisting of Melissa officinalis, hybericum, Camellia sinensis, Aronia melanocarpa, Emblica officinalis, Olea Europa, Citrus bergamia, Vaccinium macrocarpon, Myrciaria dubia, red Panax ginseng, Vaccinium oxycoccos, Vaccinium macrocarpon.

A further aspect is the non-medical use of the composition comprising pasteurized Akkermansia for reducing anxiety, stress or non-depressive mood disorders or for promoting mental health in IBS patients.

A further aspect is a method of treatment of anxiety, stress or non-depressive mood disorders in IBS patients, comprising administering a composition comprising pasteurized Akkermansia to patients suffering from IBS.

### DEFINITIONS

“Treatment” means reducing or alleviating at least one adverse effect or symptom of a disease, disorder or condition. This term thus refers to both therapeutic treatment and prophylactic or preventative measures.

“Prevention” means preventing in the sense of keeping from happening or reducing the risk of a disease or condition.

“Effective amount” or “therapeutically effective amount” refers to level or amount of agent that is aimed at, without causing significant negative or adverse side effects to the target, delaying or preventing the onset of a metabolic disorder, slowing down or stopping the progression, aggravation, or deterioration of one or more symptoms of the metabolic disorder; bringing about ameliorations of the symptoms of the metabolic disorder; reducing the severity or incidence of the metabolic disorder; curing the metabolic disorder; or restoring the normal amount and/or proportion of Akkermansia muciniphila in the gut of the subject to be treated.

“Akkermansia muciniphila” refers to the mucin-degrading bacteria identified by Derrien (Derrien et al., 2004. Int. J. Syst. Evol. Microbiol. 54:1469-1476). Cells are oval-shaped, non-motile and stain Gram-negative. Akkermansia muciniphila may also be referred as Akkermansia spp. or Akkermansia-like bacteria. It belongs to the Verrucomicrobia phylum. It is generally accepted that strains with a nucleotide similarity as experimentally determined by DNA-DNA hybridization of about 70% can be considered as the same species—this corresponds to an average nucleotide identity (ANI) of approximately 95%.

“Pasteurized Akkermansia muciniphila” refers to Akkermansia muciniphila submitted to a heating treatment. In one embodiment, pasteurized Akkermansia muciniphila refers to Akkermansia muciniphila which was heated at a temperature from 50° C. to 100° C. for at least 10 minutes.

“TFU” or “Total Fluorescent Units” means the number of cells as determined by their fluorescence radiance after staining.

In a preferred embodiment, the TFU is measured by flow cytometry. For example, in a first step, aliquots of biomass batches are rehydrated in PBS and stained with Syto 9 and propidium iodide according to manufacturer protocol (LIVE/DEAD® BacLight™ Bacterial

Viability Kit, Thermofisher). The TFU may then be obtained by analyzing the stained samples on Attune NxT flow cytometer.

“Probiotics” refers to live microorganisms which, when administered in an effective amount, provide a beneficial effect on the health or well-being of a subject. In one embodiment, these health benefits are associated with improving the balance of human or animal microbiota in the gastro-intestinal tract, or restoring normal microbiota.

“Prebiotic” refers to a substance, such as, for example, a substance which may not be digested by humans, but which modulates composition and/or activity of the gut microbiota through its metabolization by microorganisms in the gut, thus conferring a beneficial physiological effect on the host.

“Subject” refers to an animal, preferably a mammal, more preferably a human or an animal.

“Anxiety” refers to a subject situation wherein the subject experiences a feeling of unease, such as worry, tensions or fear, that can be mild or severe. Typical symptoms may include increased blood pressure. In a preferred embodiment, anxiety involves experiences of intense fear and distress.

In one embodiment, anxiety is not associated with depressive mood disorder, in particular not with “major depressive disorder”.

In one embodiment, “depressive mood disorder” or “depression” involves the presence if at least one major depressive episode. A “major depressive episode” is the experience of either depressed mood or loss of interest and/or pleasure for most of every day for at least two weeks as defined in the Diagnostic and Statistical Manual of Mental disorders (DSM-IV-TR) and the International Classification of Diseases and Related Health Problems (ICD-10).

“Non-depressive mood disorder” refers to a mental disorder other than depressive mood disorder.

### DETAILED DESCRIPTION OF THE INVENTION

The applicant herein shows that the beneficial and rapid effects on IBS-related anxiety, stress or non-depressive mood disorder after administration of a composition comprising pasteurized Akkermansia muciniphila.

In to one embodiment, pasteurized Akkermansia muciniphila of the invention are non-viable cells. As used herein, “non-viable cells” means cells that are not able to proliferate. Examples to visualize or count cells of Akkermansia muciniphila have been provided by Derrien et al. (2008. Appl. Environ. Microbiol. 74:1646-8), Derrien et al. (2011. Frontiers Microbiol. 2:166-175) or Reunanen et al. (2015. Appl. Environ. Microbiol. 81 (11): 3655-62).

The composition of the invention may also comprise a pharmaceutically acceptable excipient or a food grade carrier, for example solvents, dispersion media, coatings, isotonic and absorption delaying agents and the like. For human administration, preparations should meet general safety and purity standards as required by FDA Office of Biologics standards. The present invention also relates to a medicament, medical food, food or dietary compliment comprising an effective amount of pasteurized Akkermansia muciniphila.

The present invention also relates to a method for treating or preventing a anxiety, non-depressive mood disorder or stress, wherein said method comprises administering an effective amount of pasteurized Akkermansia muciniphila.

Another object of the invention is a method for restoring a normal proportion of Akkermansia muciniphila, in the gut of a subject in need thereof, wherein said method comprises administering an effective amount of Akkermansia muciniphila to the subject.

In one embodiment of the invention, the composition of the invention is administered at least once a week, preferably at least twice a week, more preferably at least three times a week, and even more preferably at least four times a week. In another embodiment, the composition of the invention is administered at least once a day, and preferably at least twice a day.

In one embodiment, the composition of the invention is administered during 1 week, preferably during 2, 3, 4, 5, 6, 7 or 8 weeks or more or even permanently.

In one embodiment, the composition of the invention is administered for a period that lasts until the desired outcome is achieved, for example reduction of IBS-related anxiety, non-depressive mood disorder or stress.

In one embodiment of the invention, the daily dosage of Akkermansia muciniphila administered is from 1.10² to about 1.10¹⁵ TFU/day, preferably from about 1.10⁴ to about 1.10¹² TFU/day, more preferably from about 1.10⁵ to about 1.10¹¹ TFU/day and even more preferably from about 1.10⁶ to about 5.10¹⁰ TFU/day.

In another embodiment of the invention, the daily dosage of pasteurized Akkermansia muciniphila is from 1.10⁶ to about 1.10¹² cells/day, preferably from about 1.10⁸ to about 5.10¹⁰ cells/day, more preferably from about 1.109 to about 5.10¹⁰ cells/day.

The present invention also relates to the cosmetic use of pasteurized Akkermansia muciniphila for reduction of IBS-related anxiety, non-depressive mood disorder or stress.

Another object of the invention is thus a non-medical use of a composition comprising an effective amount of pasteurized Akkermansia muciniphila and the use thereof for reduction of IBS-related anxiety, non-depressive mood disorder or stress.

In one embodiment of the invention, the composition, the pharmaceutical composition, the cosmetic composition or the medicament further comprises additional probiotic strains or species, such as, for example, bacterial probiotic strains or species. In another embodiment, the further strains are pasteurized. These probiotics include bacteria, or fungal strains or species, preferably yeast strains or species. In one embodiment, said additional probiotic strains or species are selected from those naturally present in the gut of the subject, preferably in the human gut, more preferably in the gut of healthy human subjects.

Examples of bacterial probiotic strains or species that may be used in the present invention include, but are not limited to Lactobacillus, Lacticaseibacillus, Lactococcus, Bifidobacterium, Veillonella, Desemzia, Christensenella, Allobaculum, Coprococcus, Collinsella, Citrobacter, Turicibacter, Sutterella, Subdoligranulum, Streptococcus, Sporobacter, Sporacetigenium, Ruminococcus, Roseburia, Proteus, Propionobacterium, Leuconostoc, Weissella, Pediococcus, Streptococcus, Prevotella, Parabacteroides, Papillibacter, Oscillospira, Melissococcus, Dorea, Dialister, Clostridium, Cedecea, Catenibacterium, Butyrivibrio, Buttiauxella, Bulleidia, Bilophila, Bacteroides, Anaerovorax, Anaerostipes, Anaerofilum, Enterobacteriaceae, Fermicutes, Atopobium, Alistipes, Acinetobacter, Slackie, Shigella, Shewanella, Serratia, Mahella, Lachnospira, Klebsiella, Idiomarina, Fusobacterium, Faecalibacterium, Eubacterium, Enterococcus, Enterobacter, Eggerthella, Dysosmobacter, Anaerobutyricum or Anaerobacterium.

Preferred probiotic strains are Akkermansia glycaniphila, E. Halii, Lactobacillus, Lacticaseibacillus, Lactococcus, Bifidobacterium, Christensenella, Clostridium, Anaerostipes, Faecalibacterium, Eubacterium, Enterococcus, Enterobacter, Eggerthella, Dysosmobacter, Anaerobutyricum or Anaerobacterium.

Examples of prokaryote strains or species that may be used in the present invention include, but are not limited to Archaea, Firmicutes, Verrucomicrobia, Christensenella, Bacteroidetes (such as, for example, Allistipes, Bacteroides ovatus, Bacteroides splachnicus, Bacteroides stercoris, Parabacteroides, Prevotella ruminicola, Porphyromondaceae, and related genus), Proteobacteria, Betaproteobacteria (such as, for example, Aquabacterium and Burkholderia), Gammaproteobacteria (such as, for example, Xanthomonadaceae), Actinobacteria (such as, for example, Actinomycetaceae and Atopobium), Fusobacteria, Methanobacteria, Spirochaetes, Fibrobacteres, Deferribacteres, Deinococcus, Thermus, Cyanobacteria, Methanobrevibacteria, Peptostreptococcus, Ruminococcus, Coprococcus, Subdolingranulum, Dorea, Bulleidia, Anaerofustis, Gemella, Roseburia, Dialister, Anaerotruncus, Staphylococcus, Micrococcus, Propionobacteria, Enterobacteriaceae, Faecalibacterium, Bacteroides, Parabacteroides, Prevotella, Eubacterium, Bacilli (such as, for example, Lactobacillus salivarius and related species, Aerococcus, Granulicatella, Streptococcus bovis and related genus and Streptococcus intermedius and related genus), Clostridium (such as, for example, Anaerobutyricum hallii, Eubacterium limosum, Anaerobutyricum soehngenii and related genus) and Butyrivibrio.

Examples of fungal probiotic strains or species, preferably yeast probiotic strains or species that may be used in the present invention include, but are not limited Ascomycetes, Zygomycetes and Deuteromycetes, preferably from the groups Aspergillus, Torulopsis, Zygosaccharomyces, Hansenula, Candida, Saccharomyces, Clavispora, Bretanomyces, Pichia, Amylomyces, Zygosaccharomyces, Endomycess, Hyphopichia, Zygosaccharomyces, Kluyveromyces, Mucor, Rhizopus, Yarrowia, Endomyces, Debaryomyces, and/or Penicillium.

In one embodiment of the invention, the only one microbial strain or species, preferably bacterial strain or species, comprised in the composition, pharmaceutical composition, cosmetic composition or medicament is Akkermansia muciniphila.

In one embodiment of the invention, the composition, pharmaceutical composition, cosmetic composition or medicament consists of pasteurized Akkermansia muciniphila.

In one embodiment of the invention, the composition, the pharmaceutical composition, the cosmetic composition or the medicament further comprises a prebiotic.

Examples of prebiotics that may be used in the present invention include, but are not limited to polyphenols, inulin and inulin-type fructans, oligofructose, beta-glucans, xylose, arabinose, arabinoxylan, ribose, galactose, rhamnose, cellobiose, fructose, lactose, salicin, sucrose, glucose, esculin, tween 80, trehalose, maltose, mannose, mellibiose, mucus or mucins, raffinose, fructooligosaccharides, galacto-oligosaccharides, polyphenols, amino acids, alcohols, fermentable carbohydrates and any combinations thereof.

Other non-limiting examples of prebiotics include water-soluble cellulose derivatives, water-insoluble cellulose derivatives, unprocessed oatmeal, metamucil, bran, and any combinations thereof.

Examples of water-soluble cellulose derivatives include, but are not limited to, methylcellulose, methyl ethyl cellulose, hydroxyethyl cellulose, ethyl hydroxyethyl cellulose, cationic hydroxyethyl cellulose, hydroxypropyl cellulose, hydroxyethyl methylcellulose, hydroxypropyl methylcellulose, and carboxymethyl cellulose.

The composition of the invention may be administered by oral administration, rectal administration, administration via esophagogastroduodenoscopy, administration via colonoscopy, administration using a nasogastric or orogastric tube.

The composition may be administered orally as tablets, pills, capsules, soft gelatin capsules, sugarcoated pills, or dispersing tablets, effervescent tablets or other solids.

The composition may be administered orally as a liquid, a drinkable solution or a liposome.

In one embodiment, the composition of the invention further comprises excipients, diluent and/or carriers selected with regard to the intended route of administration. Examples of excipients, diluent and/or carriers include, but are not limited to, water, phosphate buffer saline, anaerobic phosphate buffer saline, sodium bicarbonate, juice, milk, yogurt, infant formula, dairy product, coloring agents, such as, for example, titane dioxide (E171), iron dioxide (E172) and brilliant black BN (E151); flavoring agents; thickeners, such as, for example, glycerol monostearate; sweeteners; coating agents, such as, for example, refined colza oil, soya oil, peanut oil, soya lecithin or fish gelatin; diluting agents, such as, for example, lactose, monohydrated lactose or starch; binding agents, such as, for example, povidone, pregelatinized starch, gums, saccharose, polyethylene glycol (PEG) 4000 or PEG 6000; disintegrating agents, such as, for example, microcrystalline cellulose or sodium carboxymethyl starch, such as, for example, sodium carboxymethyl starch type A; lubricant agents, such as, for example, magnesium stearate; flow agent, such as, for example, colloidal anhydrous silica, etc.

In one embodiment of the invention, the composition of the invention is a pharmaceutical composition.

In another embodiment, the composition of the invention is a food additive, drink additive, dietary supplement, nutritional product, medical food or nutraceutical composition.

The composition of the present invention may be used for restoring gut permeability, for restoring impaired mucus production, for restoring epithelium barrier, for restoring immune system, or for restoring the production of antibacterial compounds or a combination thereof.

Moreover, the composition of the invention may be useful in controlling gut barrier function or in restoring the gut-brain axis. Accordingly, another aspect is a method for controlling gut barrier function or for controlling or restoring the gut-brain axis comprising administering an effective or cosmetically effective amount of the composition of the invention comprising pasteurized Akkermansia muciniphila to a subject in need thereof.

In one embodiment, the composition of the invention regulates mucus layer thickness which may be decreased in IBS.

In another embodiment, the administration of the composition of the invention induces the production of colon antimicrobial peptides, such as, for example, Reglllgamma.

In another embodiment, the administration of composition of the invention induces the production of compounds of the endocannabinoids family, such as, for example, acylglycerols selected from the group comprising 2-oleoylglycerol, 2-palmitoylglycerol and 2-arachidonoylglycerol.

In another embodiment, the administration of the composition of the invention regulates mucus tumover.

In one embodiment, the administration of the composition of the invention to a subject increases mental health, reduced stress or anxiety in IBS-patients. Consequently, according to this embodiment, the method of the invention may increase mental health or may decrease stress, anxiety or non-depressive mood disorders.

### SHORT DESCRIPTION OF THE DRAWINGS

FIG. 1 shows the effects of two doses of pAkk on number of the entry in open arm in Elevated Plus Maze (EPM) in C57BL/6J mice after clearance of C. rodentium challenge. Elevated plus maze tests were conducted after 5 days of pAkk administration to study anxiety-like behaviors (NI: Non-infected mice; Citro: C. rodentium infected groups; pAkk High dose: pasteurized A. muciniphila at high dose; pAkk Low dose: pasteurized A. muciniphila at low dose). Values are represented as mean±SEM. * p<0.05, **** p<0.0001 (p values were obtained using 1-way ANOVA followed by Dunnett's post-hoc test.

FIG. 2 shows the effects of two doses of pAkk on time spent in open arm in Elevated Plus Maze (EPM) in C57BL/6J mice after clearance of C. rodentium challenge. Elevated plus maze tests were conducted after 5 days of pAkk administration to study anxiety-like behaviors (NI: Non-infected mice; Citro: C. rodentium infected groups; pAkk High dose: pasteurized A. muciniphila at high dose; pAkk Low dose: pasteurized A. muciniphila at low dose). Values are represented as mean±SEM. ** p<0.01, **** p<0.0001 F′ (p values were obtained using 1-way ANOVA followed by Dunnett's post-hoc test

### DETAILED DESCRIPTION OF THE DRAWING

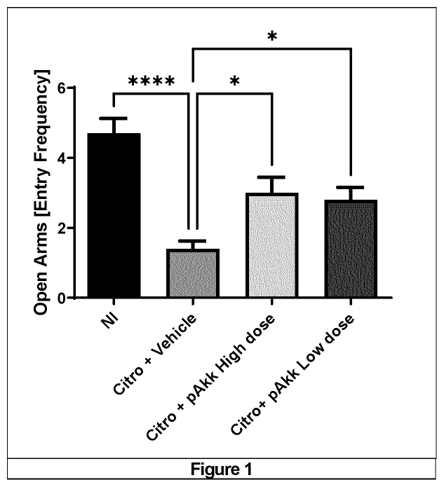

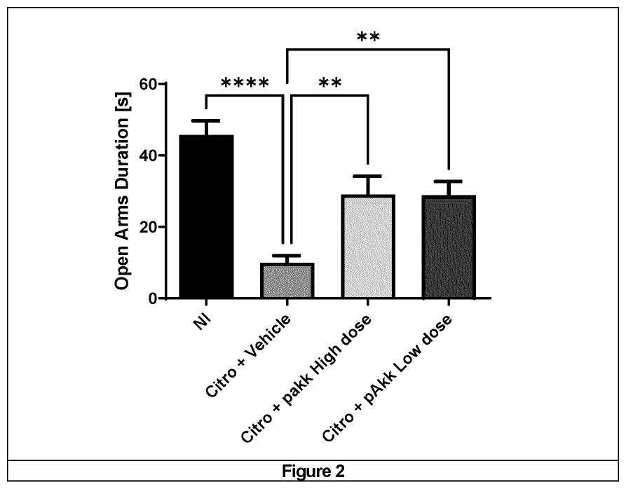

FIGS. 1 and 2 show the in vivo effect of pasteurized Akkermansia muciniphila (in the Figures referred to as “pAkk”) on anxiety-like behaviour in mice previously challenged with Citrobacter rodentium hereinafter referred to as “Citro”.

Seven-week-old C57BL/6J male mice were purchased from Janvier Lab. (France). One week after acclimatization mice were infected with C. rodentium (1×10⁹ CFU/mice in 200 μL of PBS). After the screening of C. rodentium infection resolution (16 days post infection), mice were treated daily by oral gavage during eight days with 250 μL of pAkk at 6.05×10⁺⁰⁸ TFU/mouse/day or 3.03×10⁺⁰⁹ TFU/mouse/day (n=10) or vehicle (n=10). In addition, a non-infected group (n=10) was also treated with the vehicle.

| Group number | Group name | Diet | Treatment |
|---|---|---|---|
| 1 | Non-infected (NI) | Normal chow diet | Vehicle |
| 2 | Citro + Vehicle | Normal chow diet | Vehicle |
| 3 | Citro + pAkk | Normal chow diet | 3.03 × 10⁺⁰⁹ |
| | High dose | | TFU/mouse/day |
| 4 | Citro + pAkk | Normal chow diet | 6.05 × 10⁺⁰⁸ |
| | Low dose | | TFU/mouse/day |

Anxiety-like behavior was assessed using the Elevated-Plus Maze (EPM) test (ViewPoint Behavior Technology, Lissieu, France) after 5 days of pAkk oral gavage. The apparatus consists of two opposite open arms (37×6×0.6 cm) and two closed arms (37×6×15 cm), joined by a common central platform (15×15 cm), and subjected to an equal illumination (30 lux). The maze is elevated 50 cm above the floor. Mice were acclimated to the room at least 45 minutes before test. Each individual animal was placed in the central zone and allowed to explore the maze for 5 minutes. Mice were recorded with a camera and data were manually scored. Anxiety is characterized through the number of entries in each arm (considered when the four taws are located within the arm) and time spent in open arms were quantified.

On day 21 (after 5 days of treatment), the anxiety-like behavior was evaluated in each mouse using the EPM reference test. If the mouse enters more in the open arms or spends more time in the open arms, decreased anxiety-like behavior is concluded. A significant decrease of the entry frequency and spent time in open arms was observed in the “Citro+CTRL” group in comparison to the non-infected group (NI) showing that infected mice exhibited an anxiety-like behavior. Surprisingly, this anxiety-like behavior is significantly reversed by both doses of pAkk tested only after 5 days of treatment.

Translation of the English expressions in the drawings:

| | |
|---|---|
| English expression | Translation |

Non-infected (NI)
Citro + Vehicle
Citro + pAkk High dose
Citro + pAkk Low dose
Non-infected (NI)
Normal chow diet
Vehicle
TFU/mouse/day
Open arms duration
Entry frequency

1. Composition comprising pasteurized Akkermansia for use in the prevention or treatment of anxiety, stress or non-depressive mood disorders, wherein the anxiety, stress or non-depressive mood disorders are related to Irritable Bowel Syndrome (IBS).

2. Composition according to claim 1, wherein the pasteurized Akkermansia is Akkermansia muciniphila.

3. Composition according to claim 1, wherein the early stage of anxiety, stress or non-depressive mood disorder are treated or prevented.

4. Composition according to claim 1, wherein the early stage is the onset of anxiety, stress or non-depressive mood disorder.

5. Composition according to claim 1, wherein the composition is administered sufficiently in advance of a stress-provoking event to prevent, control or reduce the formation of anxiety, stress or non-depressive mood disorder to a subject.

6. Composition according to claim 1, wherein pasteurized Akkermansia is administered for 2 to 10 days, preferably for 3 to 7 days and even more preferably for 4 to 6 days.

7. Composition according to claim 1, wherein the pasteurized Akkermansia is administered in an amount from 1.10⁴ to 1.10¹² cells per day, more preferably from 1.10⁵ cells to 1.10¹¹ cells per day, and even more preferably from 1.10⁶ to 5.10¹⁰ cells per day.

8. Composition according to claim 1, wherein the pasteurized Akkermansia is administered in an amount from 1.10⁸ to 5.10¹⁰ cells per day.

9. Composition according to claim 1, further comprising one or more ingredients chosen from group consisting of probiotic, bacteria, yeast, microorganisms, prebiotic, or a combination thereof.

10. Composition according to claim 1, wherein the composition further comprising a mineral or a vitamin or a combination thereof.

11. Composition according to claim 1, wherein the composition further comprises a pharmaceutically acceptable carrier or a food grade carrier.

12. Composition according to claim 1, wherein the composition is orally administered.

13. Composition according to claim 1, wherein the composition is a cosmetic composition, a nutritional composition, a food product, a dietary complement, a medical food, or a medicament.

14. Composition according to claim 1, wherein the composition further comprises a plant extract, chosen from the group consisting of Camellia sinensis, Aronia melanocarpa, Emblica officinalis, Olea Europa, Citrus bergamia, Vaccinium macrocarpon, Myrciaria dubia, red Panax ginseng, Vaccinium oxycoccos, Vaccinium macrocarpon.

15. Non-medical use of the composition according to claim 1, for reducing anxiety, stress or non-depressive mood disorders or for promoting mental health in IBS patients.
